(19) United States
(12) Patent Application Publication
(10) Pub. No.: US 2004/0236247 A1
(43) Pub. Date: Nov. 25, 2004

(54) Endocervical Curette

(76) Inventor: Syed Rizvi, Bakersfield, CA (US)

Correspondence Address: JEFFREY FURR, 253 N. MAIN STREET, JOHNSTOWN, OH, 43031, US

(21) Appl. No.: 10/249,990

(22) Filed: May 24, 2003

(51) Int. Cl. A61B 10/00

(52) U.S. Cl. 600/569

(57) ABSTRACT

A device for scrapping and collecting the tissue from body cavities like endocervix to perform tissue biopsy procedures such as endocervical curettage. The instrument includes a screw shape scraper and a brush mounted to the either ends of an elongated handle shaft. The scraper has a screw shape with blunt end mounted on one end of a shaft. The other end of the shaft has a brush with brissels to facilitate the trapping and collection of the tissue. The device is disposable and made of plastic as much as possible.

### BACKGROUND OF INVENTION

The present invention relates to a medical device that provides for better insertion and the sampling of tissue from body cavities, particularly the female cervix. This invention relates generally to an endocervical curette useful for detecting cervical cancer beyond the site of the colposcope. More specifically, the present invention relates to an improved device to obtain adequate tissue samples from inside the cervix. Thus assuring more accurate diagnosis of cervical pathology.

### PRIOR ART

The detection of abnormal cell growth in the cervix is important for detecting a number of disease conditions from precancerous dysplasia to invasive cancer. This process begins by first obtaining a Pap smear of the cervix. Pap smear is a screening method for cervical cancer where physician takes a swab from the cervix and places it on a slide. The slide is then examined by a pathologist who determines if there is an abnormality requiring further evaluation. If the Pap smear is abnormal, physician usually evaluates the patient further. The physician typically uses a colposcope, which magnifies the view to identify and examine suspicious areas of the cervix and takes biopsies from these areas. If the upper areas of the cervix at or beyond the internal os are involved, the physician will typically take a blind biopsy using a curette to perform endocervical curettage (ECC). The ECC procedure is very important for timely and accurate diagnosis of cervical abnormalities. Some physicians routinely perform endocervical curettage during colposcopic examination of the cervix even if no lesion is visible since the lesions may be present in the endocervix, which is the area outside of the viewing capability of the colposcope. Routine endocervical curettage during colposcopy can assist physicians in detecting skip lesions inside the endocervical canal. Endocervical curettage results are pivotal in determining management choices for the patient.

The cervix presents problems for the physicians, particularly in the area up to the anatomical internal os. This is an area that it is difficult for the physician to observe directly even with the aid of instruments, rendering biopsy sampling and curettage problematic. A significant number of lesions detected by a Pap smear occur in the internal os, beyond the area seen in a colposcopic examination.

In addition, every cervix differs in size according to a number of factors such as age and the number of births. The insertion of prior art ECC devices typically involves dilating the cervix using a separate dilator. This procedure can vary from uncomfortable to painful. Frequently the dilator used must be larger than the ECC device because the dilator must be removed and the endocervical curette inserted. The time involved increases the period of time patient endures discomfort or pain.

For some women the pain is significant enough that they refuse an ECC despite the risk of missing an invasive cervical cancer. It is very important that the physician give the pathologist adequate material to make possible a correct diagnosis for relatively hidden or obscure areas so that the patient is not subjected to a cone biopsy, which is extremely dangerous both at the time and in future reproductive years when, among other problems, mid-trimester abortions occur. Early dysplasia and/or carcinoma occurs even at young age, so every means should be taken to diagnose and treat this slow-moving disease before it becomes invasive. The results of missed diagnosis can be from debilitating to fatal. Once invasive, the consequences are devastating. Treatments like chemotherapy, radiation or radical surgery, even if successful in stopping the cancer, can still result in loss of several organ functions and psychological abnormality leaving patients sexually crippled and/or sterile.

U.S. Pat. No. 4,340,066 to Shah describes an instrument having a longitudinal chamber with a slot and a transverse slot. The handle is connected to a syringe and the physcian uses the entire probe to scrape and gather tissue samples.

U.S. Pat. No. 4,393,879 to Milgrom discloses a curette with a tissue-scraping device such as a spoon at one end. The handle member is operated by utilizing both hands to produce a sucking action to collect the tissue samples.

U.S. Pat. No. 4,627,444 to Booker describes a device for sampling tissues and fluids from body cavities.

The endocervical curettes currently available in the market have following problems:

1. The curettes do not easily penetrate the stenotic cervix commonly seen among menopausal females.

2. Procedure with these curettes is painful and difficult to tolerate by patients. Patients commonly scream and cry during the procedure.

3. Inability to obtain adequate endocervical material with these curettes for tissue diagnosis without significant pain and discomfort.

4. The tissue is difficult to dislodge from the curette due to the lack of appropriate dislodging mechanism.

5. Do not provide a circular cutting mechanism in the endocervix thus potentially missing the lesions.

6. Lack of circular cutting of the endocervical tissue prevents from getting specimen in adequate quantity.

7. Do not dilate the cervix.

8. Do not have adequate trapping mechanism for the tissue.

9. Require separate instrumentation for cervical dilatation and endocervical curettage.

10. Require sterilization and maintenance of related paper work, chemicals, supplies, equipment, policies and procedures, which adds up to the cost of the procedure.

11. Carry a risk of potential infection if not properly sterilized.

12. Cost prohibits stocking the facility with needed number of instruments without going through frequent sterilization process.

13. Carry a risk of occupational hazard for support personnel handling the instrument after the procedure.

### SUMMARY OF INVENTION

It is an object of the present invention to provide a device overcoming above described problems associated with the use of endocervical curettes currently available in the market.

Another object is to provide one implement, which will easily dilate, penetrate the stenotic cervix and obtain biopsy sample.

A further object is to provide an implement with all the needed mechanism like cervical dilation, cutting, tissue trapping, tissue collecting and tissue dislodging built into one instrument.

A still further object of the invention is the provision of a device that is less painful for the patients and provides for adequate sampling of the tissue.

Still another object is to provide an implement with proper tissue trapping and tissue dislodging mechanism for easy collection in appropriate quantities.

Yet another object of the present invention is the provision of a circular cutting mechanism in the endocervix to improve the sampling.

Another object is to provide an implement, which may be used as a disposable device. A still further object is to decrease the cost and labor associated With the maintenance of instruments related to the endocervical curettage.

Other object is to decrease the risk of occupational hazard to the medical personnel.

A further object is to eliminate the risk of iatrogenic hazard and infection to the patient.

The current invention has a tube within a tube, with a retrieval line, multiple cutting edges, a protective sleeve, a stop sleeve, a plug and a cap. The catheter has integral parts and appears to be a rather difficult instrument to use. After insertion, the protective cap or tip is dislodged into the patient and the probe or curette includes a plurality of notches for blindly scraping tissue samples from the cavity walls. A syringe is attached and provides suction to collect the scraped tissue samples.

Additional objects and advantages of the invention will be set forth in part in the description, which follows, and in part will be obvious from the description, or may be learned by practice of the invention.

### BRIEF DESCRIPTION OF DRAWINGS

The drawing, which is incorporated in and constitute a part of this specification, illustrate a preferred embodiment of the invention and, together with the description, serve to explain the principles of the invention.

FIG. 1 is a drawing of the current invention.

### DETAILED DESCRIPTION

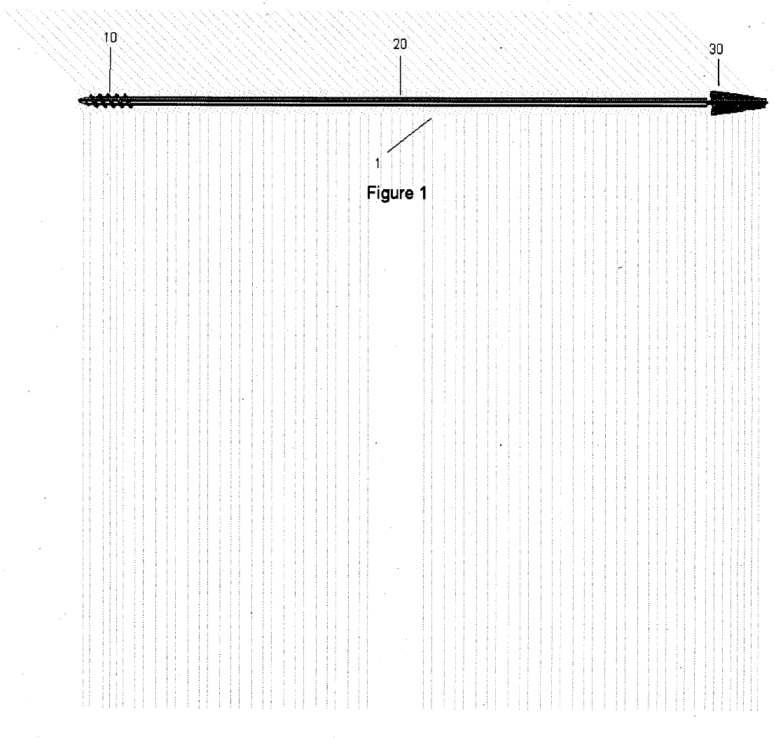

FIG. 1 is a perspective view of the device 1 for scrapping and collecting the tissue from body cavities. Said device comprises of a shaft 20 having a distal end and a proximal end. A scrapper 10 at one end and a brush 30 at the other end. The shaft is approximately 23 centimeters long and 3 millimeters wide. The brush is approximately 2 centimeter long and gradually decreases in width from approximately 8 millimeters to 5 millimeters. The scrapper 10 is approximately 2 centimeter long and gradually decreases in width from approximately 4-5 millimeters to 2 millimeters. These dimensions can vary and the scrapper 10 may or may not be tapered to accommodate the specific needs of the users. Surface of the projections can be straight or concave on the sides to be able to hold the dissected tissue.

The scrapper 10 is a screw shape part of the device 1 with a blunt end. The screw shape of the scrapper 10 is unique assuring that the device is inserted into the endocervix easily by rotating the device in clockwise direction. This shape also provides for dissection of the tissue in circular fashion throughout the entire endocervical canal. The back and forth motion of the scrapper 10 assures that the tissue from entire endocervical canal, crypts and folds is dislodged for collection. This shape provides for easy dilatation of a stenotic cervix by rotating the device 1 in clockwise direction.

The screw shape of the device 1 facilitates dislodging the specimen after the biopsy by placing the device 1 on a specimen collection pad and rotating the curette in anti-clockwise direction. Holding the pad firmly around the device 1 and rotating the device 1 in anticlockwise direction assures that the entire specimen is dislodged and placed on the specimen pad, which is then sent to pathology for evaluation. Alternatively the device 1 can be placed in a specimen collection fluid, moved and rotated to dislodge the specimen.

In the preferred embodiment, the device 1 is colored or marked for orientation and determining the location inside the body organ.

The scrapper 10 part of the device 1 is introduced into the endocervix and rotated in clockwise direction followed by the back and forth movement. The device 1 is then removed and the scrapper 10 is placed on the specimen collection pad. Holding the pad firmly around the scrapper 10 and rotating the device in anti clockwise direction dislodges the tissue. Next the brush 30 part of the device 1 is introduced into the endocervical canal, rotated as needed to trap the tissue, removed and placed on the specimen collection pad. The pad is held firmly around the brush 30 and the device 1 is pulled outwards to collect the tissue onto the pad. The specimen pad is then sent to pathology for evaluation. Alternatively the scrapper 10 and/or the brush 30 can be placed in a specimen collection fluid, moved and rotated to dislodge the specimen.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

It is to be understood that the present invention is not limited to the embodiments described above, but encompasses any and all embodiments within the scope of the claims.

It is to be understood that the foregoing description is exemplary and explanatory but are not restrictive of the invention.

What is claimed is:

1. A device for scrapping and collecting the tissue from body cavities, said device comprising: a shaft having a distal end, and a proximal end; with a scrapper at one end and a brush at the other end.

2. A device of claim 1 wherein said scrapper is in a screw shape.

3. A device of claim 1 wherein said scrapper is in a screw shape and can servce as a dilator.

4. A device of claim 1 wherein said scrapper includes: A first plurality of screw shape projections defining first predetermined dimensions, and another plurality of projections defining dimensions different than first plurality of projections.

5. A device of claim 1 wherein said device is straight about an imaginary center-line plane.

6. A device of claim 1 wherein said scrapper is located on the opposite side of the shaft as said brush.

7. A device of claim 4 wherein each of the said projections are round in shape

8. A device of claim 4 wherein said projections are screw shape and circular.

9. A device of claim 4 wherein said projection on the tip is smaller than the projection at the opposite end for easy introduction into the endocervix.

10. A device of claim 4 wherein said projections gradually differ in size.

11. A device of claim 4 wherein said projections have a predetermined distance between the projections creating a space to entrap the dissected tissue.

12. A device of claim 4 wherein said projections have sharp edges to facilitate tissue dissection.

13. A device of claim 4 wherein said projections on the scrapper have a predetermined distance between the projections to allow advancement into the endocervix by clockwise rotation.

14. A device of claim 4 wherein said projections on the scrapper have a predetermined distance between the projections to allow backward movement out of the endocervix by anticlockwise rotation.

15. A device of claim 1 wherein said shaft provides for attachment of said scrapper and said brush.

16. A device of claim 4 wherein said projections gradually differ in size for easy cervical dilatation.

17. A device of claim 4 wherein said projections have a straight surface on the side to be able to hold the dissected tissue.

18. A device of claim 4 wherein said projections have a concave surface on the side to be able to hold the dissected tissue.

19. A device of claim 4 wherein said projections have a conventional surface.

20. A device of claim 1 wherein said shaft provides for removal and attachment of said scrapper and said brush.
